(19) United States
(12) Patent Application Publication
(10) Pub. No.: US 2018/0028214 A1
(43) Pub. Date: Feb. 1, 2018

(54) TENSIONED COLLET SCALPEL HANDLE

(71) Applicant: Friedman Medical Instruments, LLC, Los Angeles, CA (US)

(72) Inventors: David H. Friedman, Los Angeles, CA (US); Steve Bond, Camarillo, CA (US)

(21) Appl. No.: 15/224,449

(22) Filed: Jul. 29, 2016

(51) Int. Cl. A61B 17/3213

(52) U.S. Cl. CPC A61B 17/3213

(57) ABSTRACT

A scalpel handle having knurling on the round shaft in close proximity to the surgical blade for improved precision, improved control, improved safety and reduced hand fatigue. The blade is secured by tension within the collet and is adjustable in length to provide a wide range of exposed positions for use. Embedded flat ruler markings enable accurate measurement of lesions or other tissue areas within the surgical field.

### CROSS-REFERENCE TO RELATED APPLICATIONS

NOT APPLICABLE

### STATEMENT REGARDING FEDERALLY SPONSORED RESEARCH OR DEVELOPMENT

NOT APPLICABLE

### REFERENCE TO SEQUENCE LISTING, A TABLE, OR A COMPUTER PROGRAM LISTING COMPACT DISC APPENDIX

NOT APPLICABLE

### STATEMENT REGARDING PRIOR DISCLOSURES BY THE INVENTOR OR A JOINT INVENTOR

NO DISCLOSURE

### BACKGROUND OF THE INVENTION

Field of the Invention

The present invention relates generally to surgical knives and more particularly to a scalpel handle for disposable surgical blades. Most particularly the invention relates to a tensioned collet scalpel handle having a rounded shaft with knurling in close proximity to the blade to provide improved precision, greater control, increased safety, and to reduce the hand fatigue associated with surgical procedures. The present invention incorporates embedded flat ruler markings for more precise and accurate measurement of areas within the surgical field.

Description of Related Art

Scalpels are among the most commonly used tools in modern surgery, yet the existing designs are frequently inadequate for curved, circular or elliptical incisions associated with dermatologic procedures. Furthermore, the poor ergonomics and design of existing scalpels force surgeons into uncomfortable and awkward positions during procedures that can negatively impact patient safety and recovery.

One of the most commonly utilized scalpels by surgeons is marketed by Becton-Dickinson Company of Franklin Lakes, N.J., under the trademark Bard-Parker®. The Bard-Parker® scalpel's flat handle has traditionally consisted of steel, aluminum or plastic, and permits the attachment of several different types of blades. Depending on the type of procedure and the necessary level of precision, a surgeon is able to attach different shapes of blades to the scalpel handle. This type of scalpel handle is designed either as a disposable single-use item or as an item made to be sterilized and reused. However, the completely flat handle contains a serious design flaw as it only provides a surgeon very little grip during procedures. The design also does not provide for sufficiently precise control while making an elliptical, circular or curved incision as it becomes necessary for a surgeon to rotate the scalpel during the procedure. The poor ergonomics of a flat handle prevents the surgeon from easily manipulating the scalpel and forces the surgeon to completely reposition their arm in order to make a curved, circular or elliptical incision. The ergonomic deficiencies of the Bard-Parker® scalpel can also negatively affect patient outcome, increase user safety risk and may cause greater scarring or other complications.

Another commonly utilized scalpel is the Siegel scalpel handle, marketed by Delasco Dermatologic Lab & Supply, Inc., Council Bluffs, Iowa. The Siegel scalpel handle is designed such that the tang of the blade is not embedded in the shaft. However, this design causes the surgeon to lose grip on the roundness of the shaft when the handle is gripped closer to the skin, and thus the surgeon loses the fine manipulation control the roundness of the shaft affords. Also the ruler marking on the Siegel scalpel handle is printed on the round handle, which makes it more difficult for surgeons to accurately measure areas within the surgical field. The ergonomic deficiencies of the Siegel scalpel can also negatively affect patient outcome, increase user safety risk and may cause greater scarring or other complications.

Another commonly utilized scalpel is marketed by Beaver-Visitec International, Inc., Waltham, Mass. under the trademark Beaver®. The Beaver® scalpel handle is designed such that the tang is embedded in the shaft. While this design enables the roundness of the shaft to be closer to the patient's skin to permit better manipulation and control, surgeons are required to use proprietary Beaver blades that are not as universally available and are not interchangeable with the much more common handles that accept the Bard-Parker® blades. Also, some surgeons have found that the ergonomics of the smaller circumference of the Beaver® scalpel handles are too small to provide sufficiently fine control. Beaver® scalpel handles also do not contain a flat ruler marking to enable a surgeon to more accurately measure areas within the surgical field.

Dermatology-related surgical procedures routinely require curved, circular or elliptical incisions, and the design shortcomings of currently available scalpels become most apparent during the surgical removal of skin lesions. This surgical procedure involves cutting an ellipse or circle around the lesion, often excising additional tissue if the lesion is suspected to be cancerous in order to ensure clear margins. After the desired amount of skin and tissue is removed, the excision is sutured together, ideally leaving a small scar. Surgeons often cut the skin at an angle to create a mirrored “V” incision to reduce scar formation. Then, the tops of each side of the “V” incision are sutured together. However, the poor ergonomics and design of currently available scalpels do not easily permit surgeons to produce completely mirrored incisions during small lesion removal. The thin and flat edges of currently available scalpels are difficult to hold and inhibit the amount of precision and control a surgeon has when making curved, circular or elliptical mirrored incisions associated with small lesion removal.

U.S. Pat. No. 5,578,050 (Webb) discloses an installable sleeve intended to enable greater traction and tactile control over a scalpel handle. The addition of a sleeve also reduces the likelihood that the scalpel would inadvertently roll in a surgeon's hand during a procedure. Although these sleeves can be molded to better fit a surgeon's hand in a more ergonomic manner than a straight scalpel handle, they are a separate component from the scalpel handle. Therefore, these sleeves are subject to torsional deformation or linear movement on the handle during a surgical procedure, which could negatively affect a surgeon's precision and a patient's outcome.

Retractable blades are found on currently available scalpels. Although this feature may reduce the risk of accidental injury caused by exposed blades by retracting the blade into the handle when not in use, retractable blade scalpels have design deficiencies that make it difficult to replace the blade. In some cases, a surgeon may have to take the entire handle apart to replace the non-sterile blade with a sterile one. U.S. Pat. No. 8,015,712 (Yi et al.) discloses a flat retractable safety scalpel having a blade, a blade holder that is fixed to the blade, a blade guard that covers the blade and within which the blade holder is able to slide, a scalpel handle that receives a blade cartridge and a locking assembly. However, ergonomic deficiencies result from having a moveable slider structure on the flat handle that may force the surgeon to grip the scalpel in an uncomfortable manner, or to adjust their preferred grip to compensate for its poor balance and reduced rigidity due to its construction. Furthermore, the flat handle's exposed channel for the adjustable slider limits the placement of knurling along the scalpel handle and in close proximity to the blade for improved grip and control during curved, circular or elliptical incisions.

U.S. Pat. No. 4,576,164 (Richeson) discloses a disposable microsurgical knife body with a cylindrical shroud in the form of a moveable sheath that can be locked into several positions in order to protect a blade located at one end of the knife body. However, this shroud may increase the diameter of the handle in certain positions and thus interfere with a surgeon's grip during procedures. Furthermore, the operation of the shroud mechanism necessitates a complex arrangement of longitudinal and circumferential grooves on the exterior surface of the knife body that can interfere with a surgeon's grip. Lastly, the presence of a moveable sheath can impact the balance and stability of the handle when utilized for curved, circular or elliptical incisions.

Surgical scalpel blades are manufactured to very precise standards and are comprised of high quality materials. For numerous reasons including infection risk, it has become customary to use a scalpel blade only once. The prior art contains several combination handle/replaceable blade scalpels as well as entirely disposable scalpels which have inexpensive plastic handles secured to the scalpel blade. With the rise of the AIDS virus and other blood-borne pathogens, much concern has arisen about the possibility of transmission of disease through contact with contaminated blood before, during or after a surgical procedure. Sharp instruments, such as scalpels that come into contact with body fluids, are particularly of concern. Although currently available scalpels attempt to improve user and patient safety both pre-procedure and post-procedure, they still contain ergonomic compromises that can negatively impact both user and patient safety during a procedure by limiting a surgeon's precision and control. Scalpels have been provided in the prior art that contain removable guards in order to prevent contact with the blade when not in use. U.S. Pat. No. 4,735,202 (Williams) discloses a scalpel having a blade guard as a slidable sleeve that is mounted over the scalpel and that may be locked into position over the blade when not in use. However, the blade guard requires two hands to move into position and the locking structure design prevents a surgeon from being able to grip the handle in closer proximity to a blade for fine control and stability during curved, circular or elliptical incisions.

Currently available scalpels also present an increased biohazard exposure risk for medical personnel during the blade disposal process. Due to the poor ergonomic design of traditional blade release mechanisms, the prior art also discloses a number of separate devices to assist with safe blade removal, such as U.S. Pat. No. 4,730,376 (Yamada), which discloses a Blade Removal Apparatus for Changeable Blade Scalpel. However, the use of separate equipment for blade removal can increase costs and also potentially increase the risk of contamination or personal injury during surgical procedures.

An improved scalpel design having a round handle with knurling in closer proximity to the blade than existing designs would enable a surgeon to more comfortably rotate and manipulate a scalpel during curved, circular or elliptical incisions. Furthermore, an improved scalpel design having a simplified blade removal mechanism that is activated at the opposite end from the blade can enhance user safety by lowering exposure risk, and eliminate the need for the use of a blade removal apparatus as disclosed in the prior art.

### BRIEF SUMMARY OF THE INVENTION

The present invention is directed at a scalpel handle for disposable surgical blades. Most particularly the invention relates to a scalpel handle having knurling on a round shaft in close proximity to the blade, a tension-adjustable blade handle and a flat ruler marking. To achieve the above objects a scalpel handle is provided with an adjustable blade that may be positioned in a range of positions within the collet by utilizing a Tensioning Jackscrew Assembly that rotates in a clockwise and counterclockwise manner.

A tensioning jackscrew has a threaded end adapted to screw into a threaded aperture of an endcap and another end adapted to mount a collet to transfer the movement of the tensioning jackscrew to the collet nodule. The endcap is a hexagonal-shaped body with a flat distal surface and its proximal surface to the tensioning jackscrew containing an aperture adapted to accept the tensioning jackscrew at the center of said endcap. When the scalpel handle is laid on a flat surface, the hexagonal-shaped body makes contact with the flat surface and functions to prevent the scalpel handle from accidental rolling movement that could result in the contamination of adjacent surgical instruments or equipment. Because the collet is keyed with a guide channel that aligns with the guide screw and guide screw marking on the shaft, the collet cannot be rotated to tighten the blade. Tightening the blade requires rotating an endcap. Rotating the endcap of the Tensioning Jackscrew Assembly in a clockwise direction draws the collet further into the shaft, thereby compressing the collet nodule against the shaft and thus increasing the tension grip of the collet on a blade for securing purposes. Rotating the endcap of the Tensioning Jackscrew Assembly in a counterclockwise direction pushes the collet out of the shaft, releases the compression of the collet nodule against the shaft and reduces the tension grip of the collet on a blade for adjustment or removal purposes. The collet guide channel, in conjunction with the guide screw and guide screw marking on the shaft, collectively works to ensure the blade maintains a fixed perpendicular orientation to the horizontal midline of the upper shaft dorsal and ventral surfaces. Maintaining the proper orientation of the blade also ensures the upper shaft ventral surface, which is a rounded structure, can roll in the first interdigital web space of the hand. Maintaining the proper orientation of the blade also ensures the upper shaft dorsal surfaces, which are flat, remain opposite and away from the first interdigital web space of the hand. These flat surfaces would otherwise not roll smoothly in the hand as the shaft is manipulated and turned to cut the skin. The knurling on the shaft in close proximity to the blade enables a surgeon to have greater level of manipulation, control and safety before, during and after procedures. An embedded ruler marking on the flat upper shaft dorsal surface enables a surgeon to have greater accuracy when measuring than a rounded scale provides. Additional ruler markings with their orientation reversed may also be embedded on other upper shaft dorsal surfaces in order to enable a surgeon to have greater flexibility when conducting measurements within the surgical field.

The improved scalpel handle may have a variety of configurations, shapes, or sizes depending on the size and type of incision required as well as the size of the surgeon's hands and fingers. In other embodiments of the invention, some or all of the individual parts that comprise the assembly may be combined, reducing the number of parts, and made of metallic materials including but not limited to aluminum or stainless steel. In other embodiments of the invention, some or all of the individual parts that comprise the assembly may be made of plastic materials to save weight or reduce cost.

The present invention introduces such refinements. In its preferred embodiments, the present invention has several aspects or facets that can be used independently, although they are preferably employed together to optimize their benefits. All of the foregoing operational principles and advantages of the present invention will be more fully appreciated upon consideration of the following detailed description, with reference to the appended drawings.

### BRIEF DESCRIPTION OF THE SEVERAL VIEWS OF THE DRAWINGS

The accompanying drawings, which are incorporated in and form a part of the specification, illustrate two embodiments of the present invention and together with the description, serve to explain the principles of the invention.

FIG. 1 is a perspective view of one preferred embodiment of the invention with the blade fully extended.

FIG. 2 is a top view of one preferred embodiment of the invention with the blade fully extended.

FIG. 3 is a bottom view of one preferred embodiment of the invention with the blade fully extended.

FIG. 4 is a side view of one preferred embodiment of the invention with the blade fully extended.

FIG. 5 is an exploded view of one preferred embodiment of the invention with the blade fully extended.

FIG. 6 is a perspective view of the Shaft Assembly of one preferred embodiment of the invention.

FIG. 7 is a second perspective view of the Shaft Assembly of one preferred embodiment of the invention.

FIG. 8 is a top view of the Shaft Assembly of one preferred embodiment of the invention.

FIG. 9 is a perspective view of the Tensioning Jackscrew Assembly of one preferred embodiment of the invention.

FIG. 10 is a side view of the Tensioning Jackscrew Assembly of one preferred embodiment of the invention.

FIG. 11 is a perspective view of the Collet Assembly of one preferred embodiment of the invention.

FIG. 12 is a second perspective view of the Collet Assembly of one preferred embodiment of the invention.

FIG. 13 is a top view of the Collet Assembly of one preferred embodiment of the invention.

FIG. 14 is a side view of the Collet Assembly of one preferred embodiment of the invention.

FIG. 15 is a second side view of the Collet Assembly of one preferred embodiment of the invention.

FIG. 16 is a perspective view of a second preferred embodiment of the invention with the blade fully extended.

FIG. 17 is a top view of a second preferred embodiment of the invention with the blade fully extended.

FIG. 18 is a side view of a second preferred embodiment of the invention with the blade fully extended.

FIG. 19 is an exploded view of a second preferred embodiment of the invention with the blade fully extended.

FIG. 20 is a perspective view of the Shaft Assembly of a second preferred embodiment of the invention.

FIG. 21 is a second perspective view of the Shaft Assembly of a second preferred embodiment of the invention.

FIG. 22 is a top view of the Shaft Assembly of a second preferred embodiment of the invention.

### DETAILED DESCRIPTION OF THE INVENTION

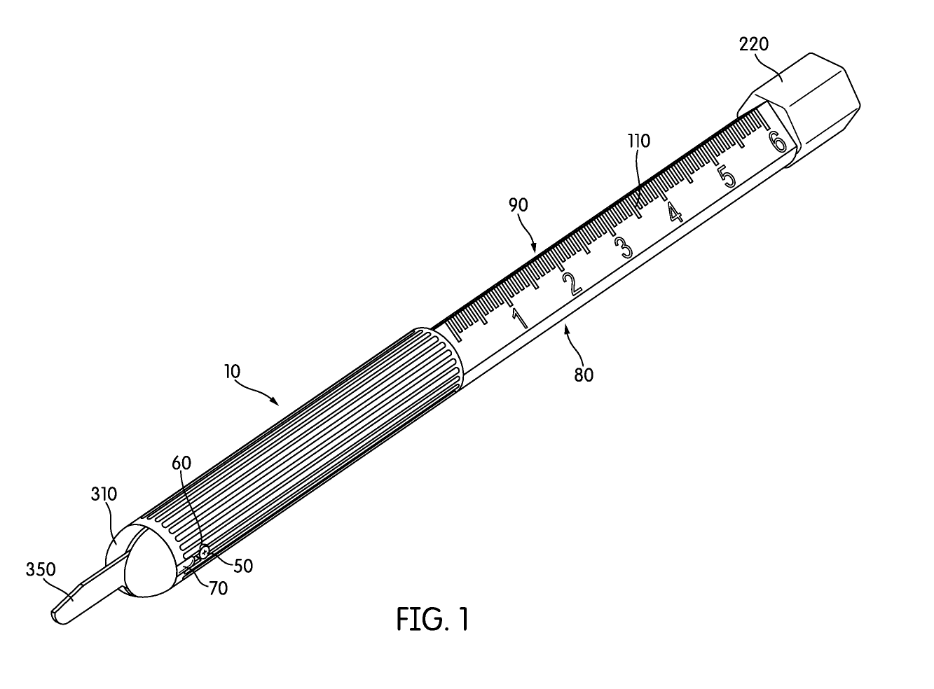

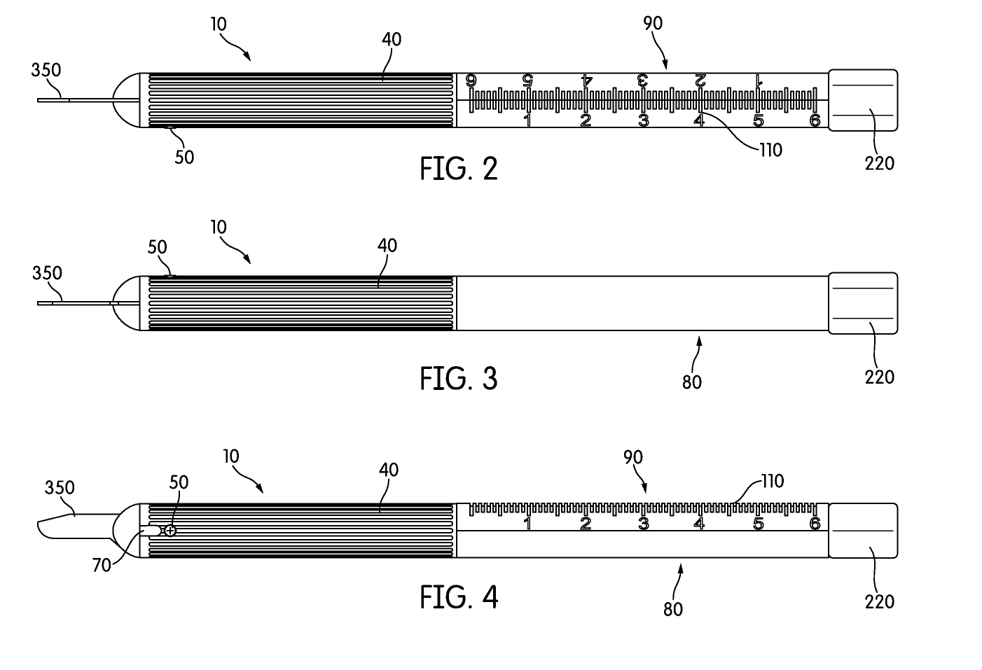

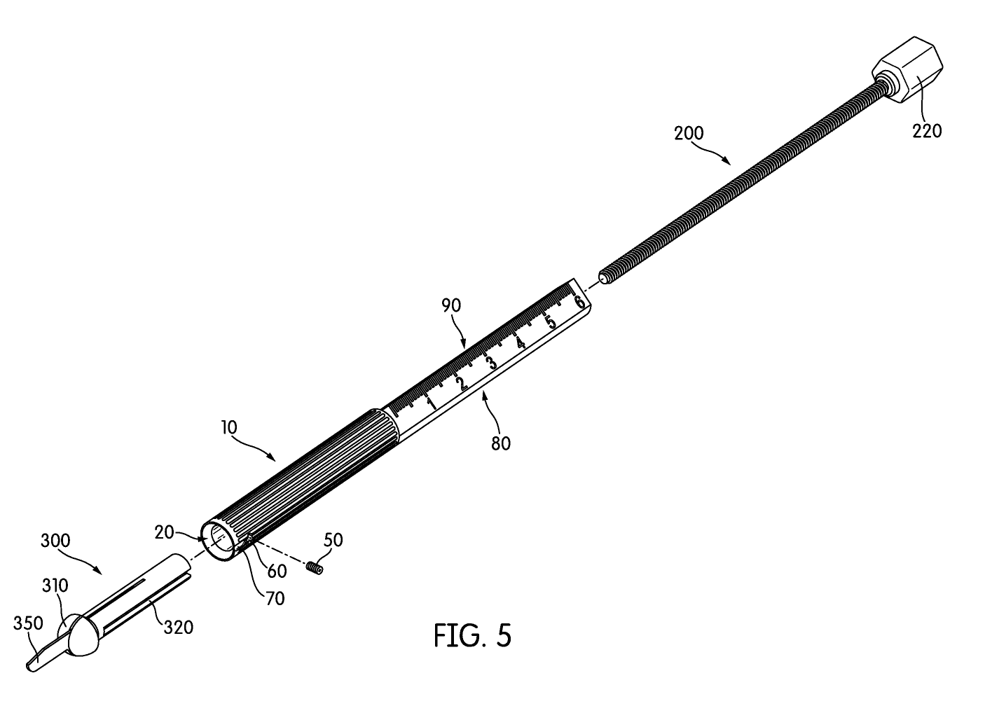

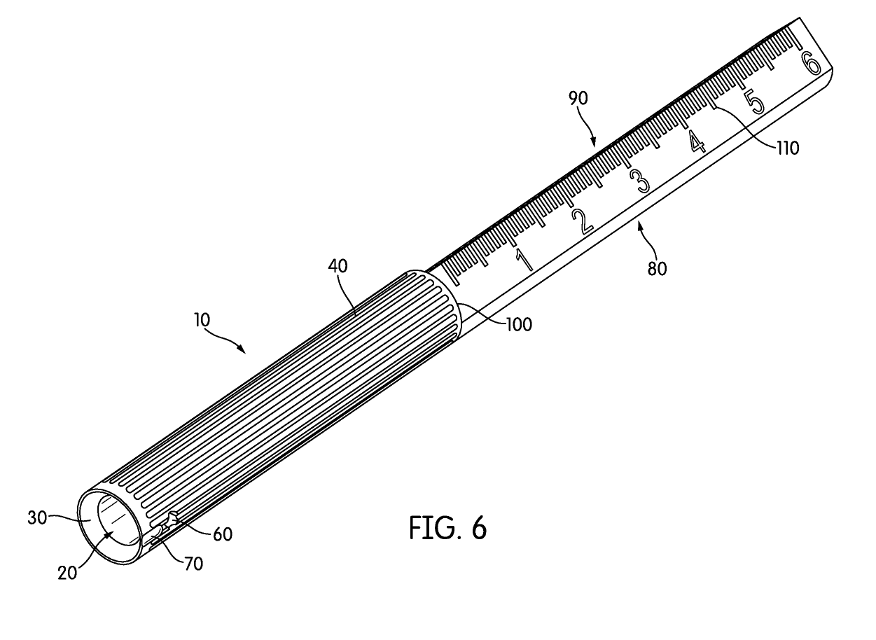

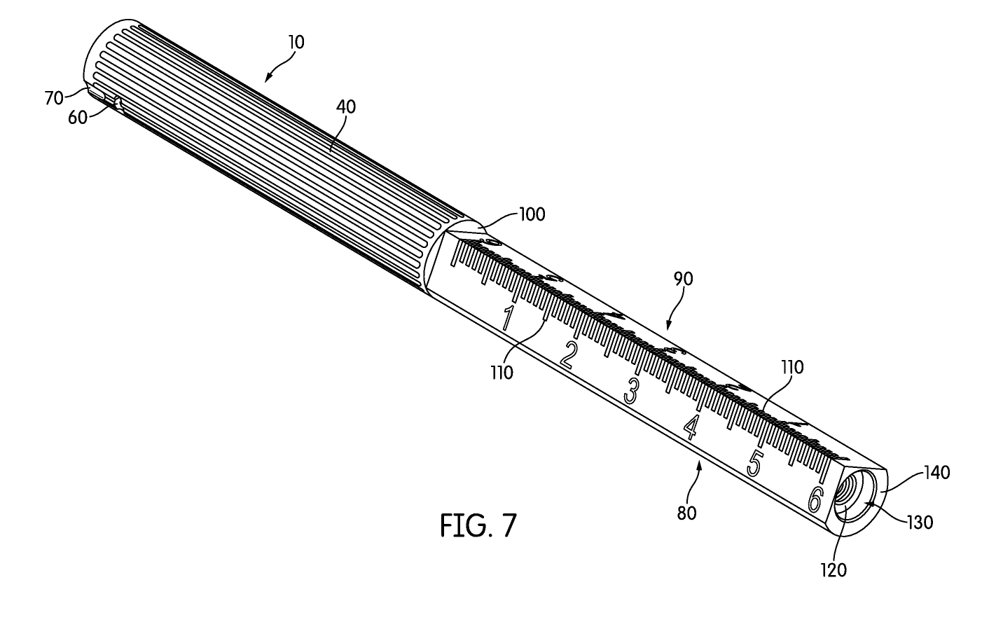

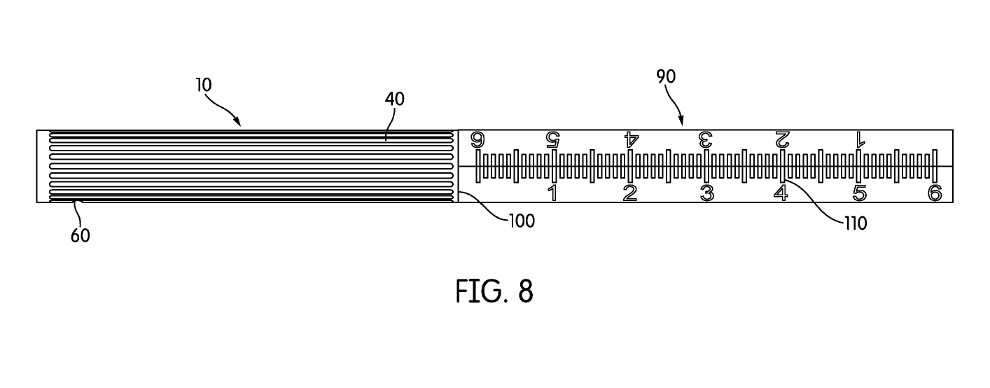

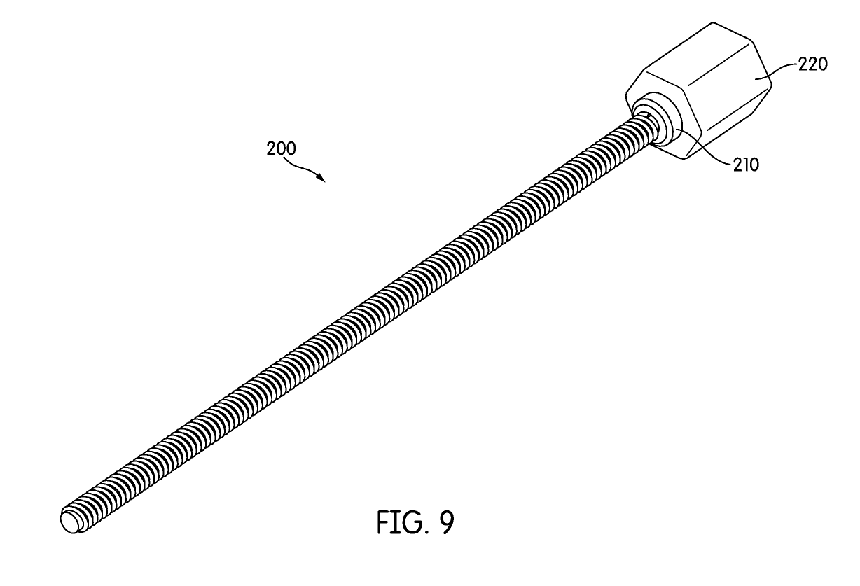

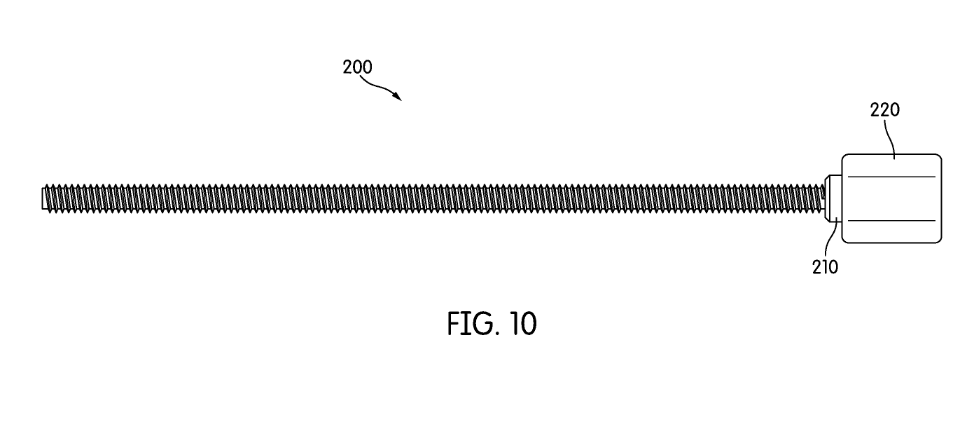

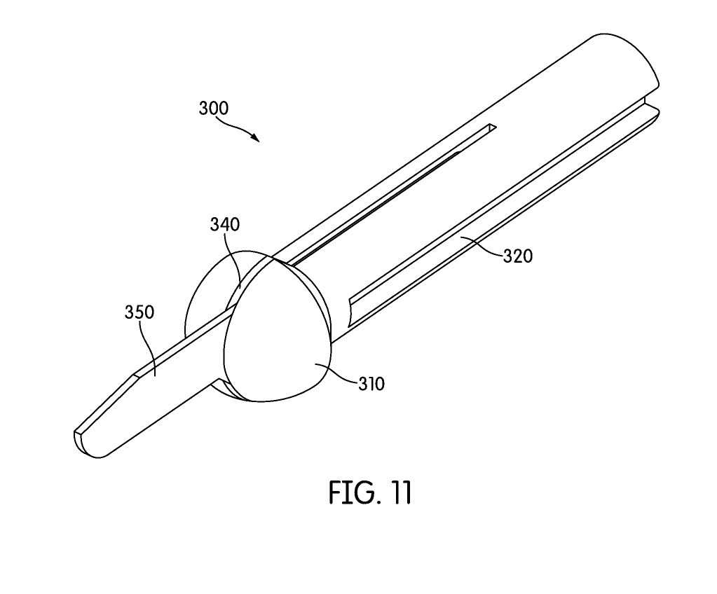

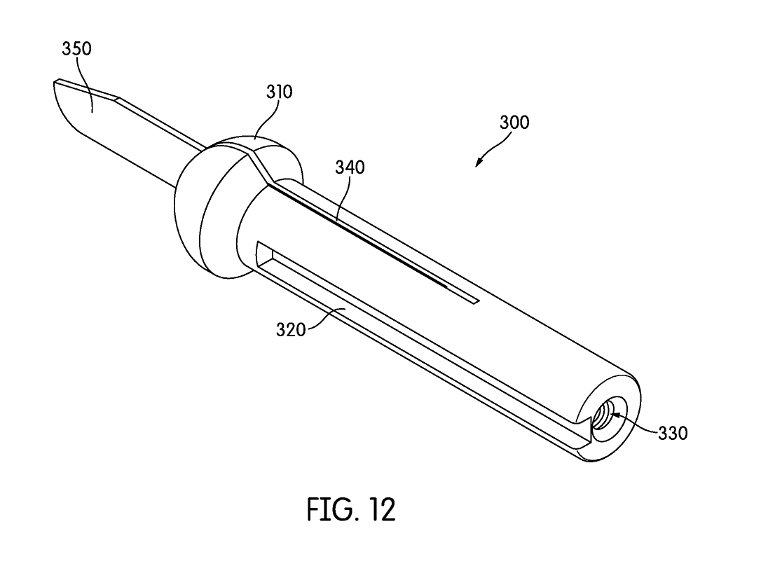

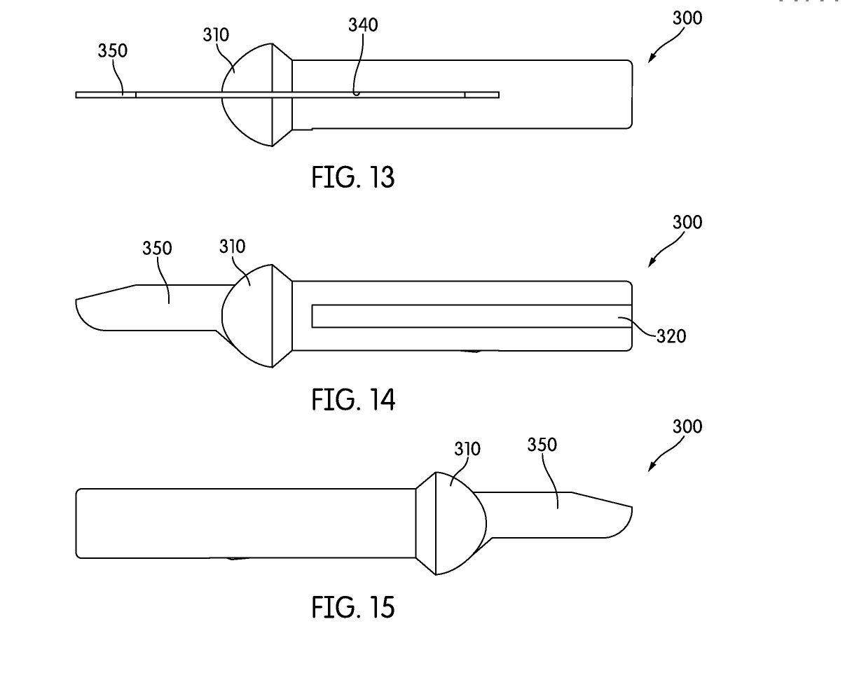

A detailed description of the present invention follows. Referring initially to FIGS. 1 through 15 of the drawings, in a first preferred embodiment the Tensioned Collet Scalpel Handle is comprised of a Shaft Assembly, Tensioning Jackscrew Assembly and a Collet Assembly. The Shaft Assembly compromises a Lower Round Shaft 10 having Knurling 40, a Guide Screw Hole 60 to receive a Guide Screw 50 and a Guide Screw Marking 70. The Shaft Assembly also comprises of a Lower Round Shaft Opening 20 having a Lower Round Shaft Inner Lip 30. The Shaft Assembly also comprises of an Upper Shaft Ventral Surface 80. The Shaft Assembly also comprises two Upper Shaft Dorsal Surfaces 90, each having a Shaft Lip 100 and a Ruler Marking 110. As disclosed in this embodiment, the ruler markings on the Upper Shaft Dorsal Surfaces are oriented in opposite directions. The Shaft Assembly also comprises an Upper Shaft Inner Lip 120 having a Tensioning Jackscrew Hole 130 and an Upper Shaft External Lip 140. The Tensioning Jackscrew Assembly comprises a threaded Tensioning Jackscrew 200 having a Jackscrew Inner Lip 210 and Endcap 220. The Collet Assembly comprises a Collet 300 having a Collet Nodule 310, a Collet Guide Channel 320 and a Threaded Collet Screw Hole 330. The Collet Assembly also comprises a Blade Receptacle 340 to receive a Blade 350.

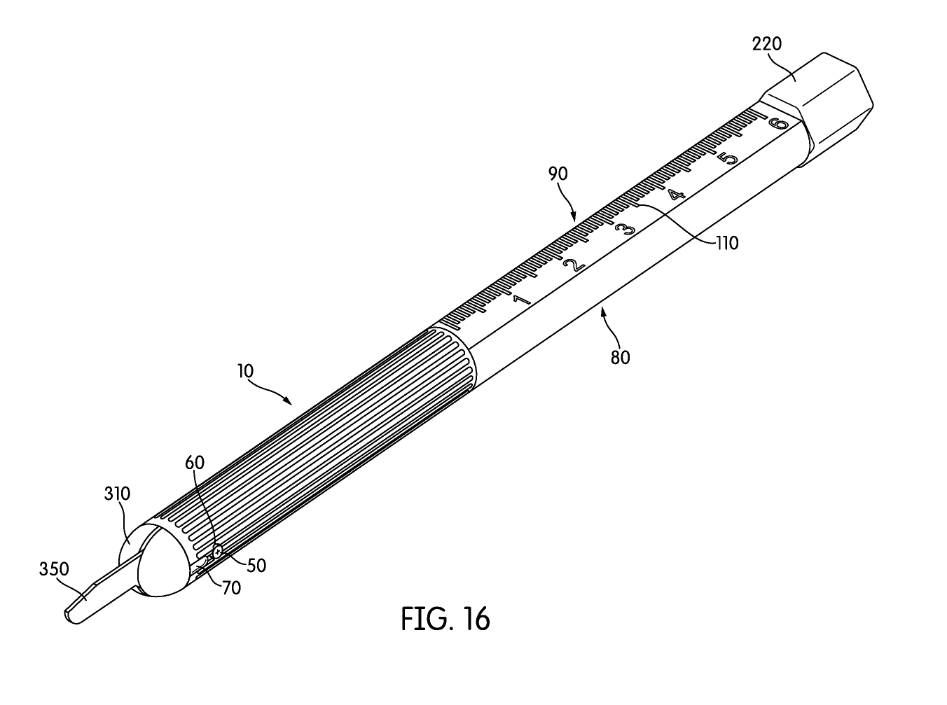

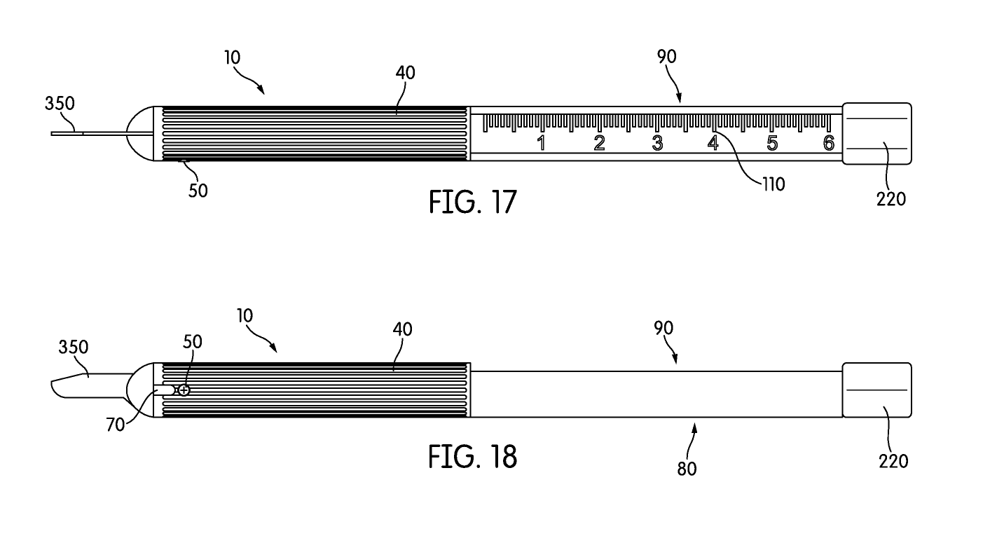

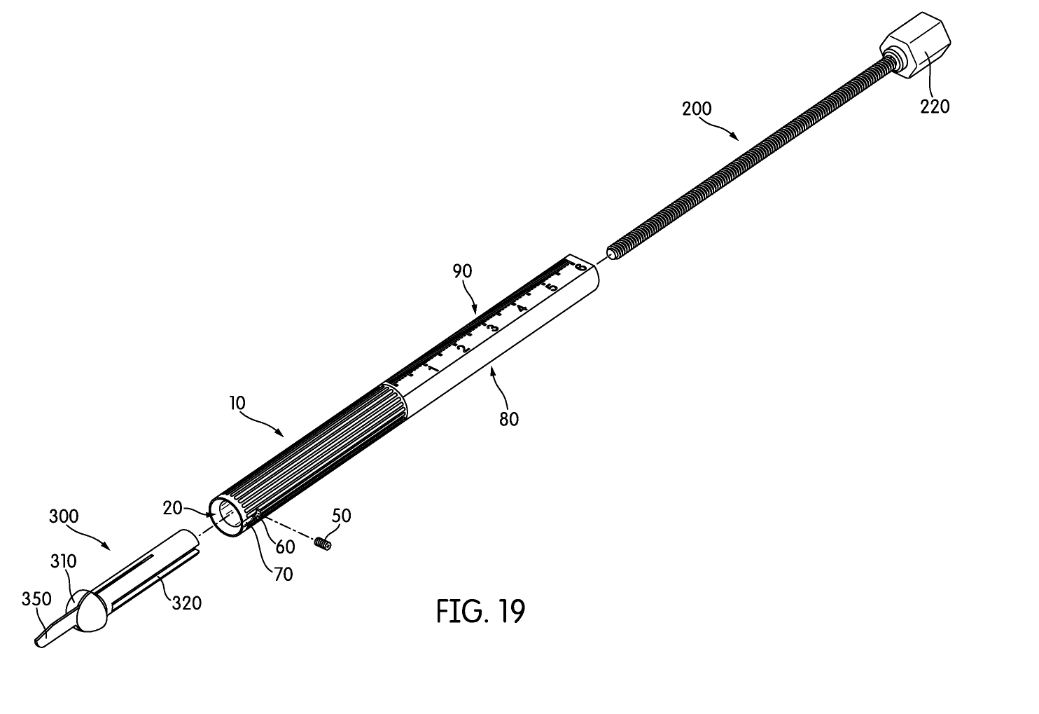

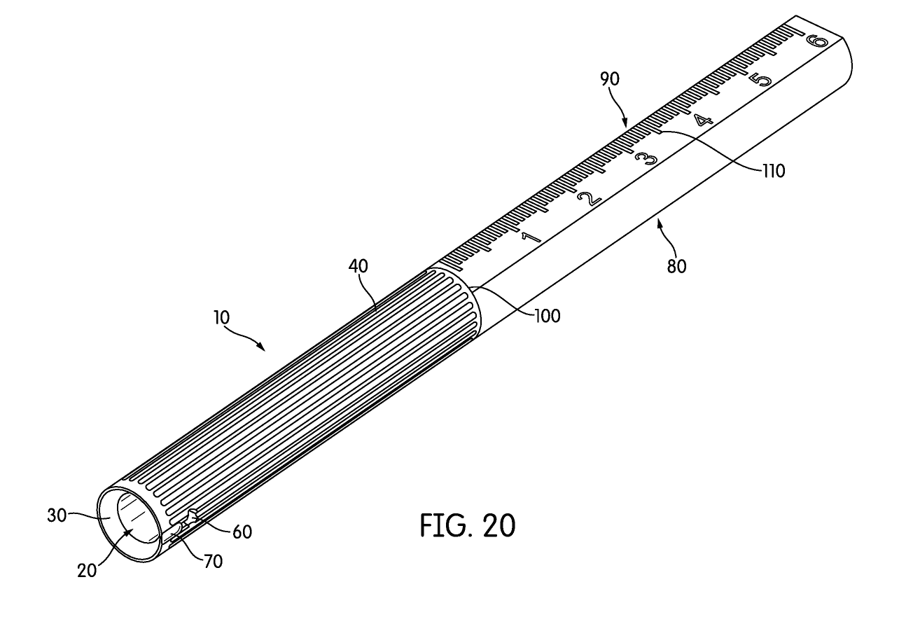

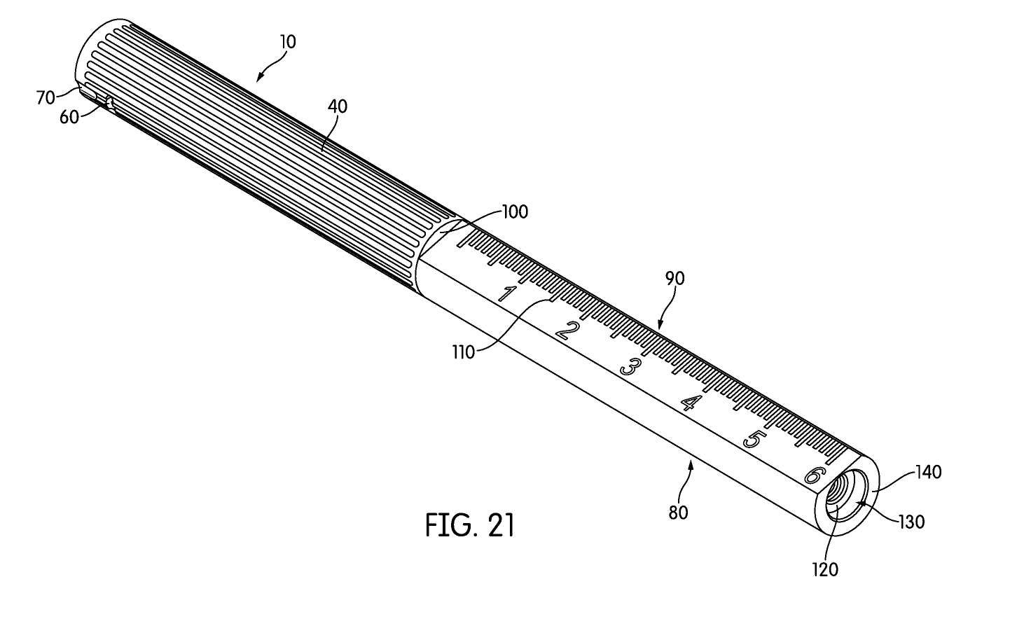

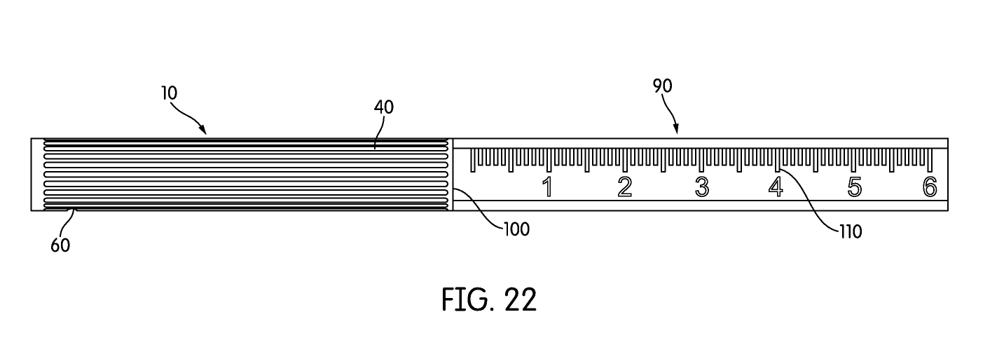

Referring next to FIGS. 16 through 22 of the drawings, which depict an alternate embodiment of the invention, the Shaft Assembly comprises an Upper Shaft Ventral Surface 80. The Shaft Assembly further comprises a single Upper Shaft Dorsal Surface 90 having a Shaft Lip 100 and a Ruler Marking 110.

In a typical application, the Tensioned Collet Scalpel Holder is initially disassembled to ensure complete sterilization into its component assemblies: Shaft Assembly, Tensioning Jackscrew Assembly and Collet Assembly.

Assembling the Tensioned Collet Scalpel Holder into a standby state involves properly aligning the Collet Guide Channel 320 with the Guide Screw Marking 70 and Guide Screw 50 at the end of the Shaft Assembly. Then, the Tensioning Jackscrew Assembly is placed at the opposing end of the Shaft Assembly, with the Tensioning Jackscrew 200 extending inside the Shaft Assembly. The Endcap 220 is then rotated in a clockwise direction to engage the threading of the Tensioning Jackscrew 200 on the Threaded Collet Screw Hole 330 on the Collet 300.

The Tensioned Collet Scalpel Holder is then brought into a ready state by placing a Blade 350 into the Blade Receptacle 340 of the Collet 300 and rotating the Endcap 220 clockwise until the Collet Nodule 310 is drawn into the Lower Round Shaft Opening 020 of the Lower Round Shaft 10. As the Collet Nodule 310 is drawn into the Lower Round Shaft Opening 20, the Lower Round Shaft Inner Lip 030 securely clenches the Collet Nodule 310 on the Blade 350.

Returning the Tensioned Collet Scalpel Holder to a standby state involves rotating the Endcap 220 counterclockwise to push the Collet 300 out of the Lower Round Shaft 10 and Lower Round Shaft Opening 20 until the Lower Round Shaft Inner Lip 30 causes the Collet Nodule 310 to release the Blade 350. The Blade 350 can then be safely removed and disposed of hands-free via gravity drop.

It can readily be seen that the above-described Tensioned Collet Scalpel Holder has many advantages over the prior art. The foregoing description of alternate embodiments of the invention has been presented for purposes of illustration and description. However, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting; but rather, to provide an understandable description of the invention. Although the invention has been disclosed in detail with reference only to the exemplary embodiments, one of ordinary skill in the art would also appreciate that each embodiment has certain advantages and it would be obvious to mix features across embodiments without departing from the scope of the invention.

The terms “a” or “an”, as used herein, are defined as one or more than one. The term another, as used herein, is defined as at least a second or more. The terms including and/or having, as used herein, are defined as comprising (i.e., open language).

Any element in a claim that does not explicitly state “means for” performing a specific function, or “step for” performing a specific function, is not to be interpreted as a “means” or “step” clause as specified in 35 U.S.C. Sec. 112, Paragraph 6.

### PARTS LISTING

### Shaft Assembly

10 Lower Round Shaft

20 Lower Round Shaft Opening

30 Lower Round Shaft Inner Lip

40 Knurling

50 Guide Screw

60 Guide Screw Hole

70 Guide Screw Marking

80 Upper Shaft Ventral Surface

90 Upper Shaft Dorsal Surface

100 Shaft Lip

110 Ruler Marking

120 Upper Shaft Inner Lip

130 Tensioning Jackscrew Hole

140 Upper Shaft External Lip

### Tensioning Jackscrew Assembly

200 Tensioning Jackscrew

210 Jackscrew Inner Lip

220 Endcap

### Collet Assembly

300 Collet

310 Collet Nodule

320 Collet Guide Channel

330 Threaded Collet Screw Hole

340 Blade Receptacle

350 Blade

The invention claimed is:

1. An apparatus for surgical procedures comprising:
a shaft assembly, a tensioning jackscrew assembly and a collet assembly;
the shaft assembly comprises:
a lower round shaft;
a lower round shaft opening;
a lower round shaft inner lip;
knurling;
a guide screw;
a guide screw hole;
a guide screw marking;
an upper shaft ventral surface;
an upper shaft dorsal surface;
a shaft lip;
a ruler marking;
an upper shaft inner lip;
a tensioning jackscrew hole;
an upper shaft external lip;
the tensioning jackscrew assembly comprises:
a tensioning jackscrew;
a jackscrew inner lip;
an endcap;
the collet assembly comprises:
a collet;
a collet nodule;
a collet guide channel;
a threaded collet screw hole;
a blade receptacle;
a blade.

2. The apparatus of claim 1, wherein said endcap is a hexagonal body.

3. The apparatus of claim 1, wherein said shaft assembly has at least one upper shaft dorsal surface which is flat.

4. The apparatus of claim 1, wherein said upper shaft dorsal surface contains at least one ruler marking.
